(19) United States
(12) Patent Application Publication
(10) Pub. No.: US 2011/0171153 A1
(43) Pub. Date: Jul. 14, 2011

(54) TREATMENT OF KERATIN FIBRES WITH VOLATILE LINEAR ALKANE(S), SOLID FATTY ALCOHOL(S) AND POLYMERIC THICKENER(S) CONTAINING SUGAR UNITS

(75) Inventors: Patricia DESENNE, Pringy (FR); Marie-Cecile Degoul, Paris (FR)

(73) Assignee: L'OREAL, Paris (FR)

(21) Appl. No.: 12/977,183

(22) Filed: Dec. 23, 2010

Related U.S. Application Data
(60) Provisional application No. 61/296,490, filed on Jan. 20, 2010.

(30) Foreign Application Priority Data
Dec. 23, 2009 (FR) 09 59485

(51) Int. Cl. A61K 8/89; A61K 8/73; A61Q 5/12

(52) U.S. Cl. 424/70.12; 424/70.13

(57) ABSTRACT

The invention relates to the use for the cosmetic treatment of keratin fibres, such as the hair, of a cosmetic composition comprising, in a cosmetically acceptable medium, more than 1% by weight, relative to the total weight of the composition, of one or more volatile linear alkanes, at least one solid fatty alcohol and at least one polymeric thickener containing sugar units.

### REFERENCE TO PRIOR APPLICATIONS

This application claims priority to U.S. provisional application Ser. No. 61/296,490, filed Jan. 20, 2010; and to French patent application 09 59485, filed Dec. 23, 2009, both incorporated herein by reference.

### FIELD OF THE INVENTION

The present invention relates to the use, for the cosmetic treatment of keratin fibres such as the hair, of a cosmetic composition comprising more than 1% by weight, relative to the total weight of the composition, of at least one volatile linear alkane, at least one solid fatty alcohol and at least one polymeric thickener containing sugar units. The described composition also makes up a part of the invention.

### BACKGROUND OF THE INVENTION

In the field of hair treatment, the use of volatile solvents is known in haircare and hair sheen products. They are generally used for various reasons. They especially make it possible to modify the sensory feel of a haircare product by giving it a light and non-tacky texture in the hand. They may also give it a slippery nature, which facilitates the spreading of the product onto the hair and in particular onto dry hair.

These volatile solvents, which are generally liquid fatty esters, hydrocarbon-based oils of isododecane or isohexadecane type, and/or silicone oils, may especially give rise to problems in terms of a greasy feel, lack of sheen, and stiff, hard hair.

There is thus still a need to replace these volatile solvents in order to avoid the drawbacks mentioned above.

### DETAILED DESCRIPTION OF THE PREFERRED EMBODIMENTS

It has now been discovered, surprisingly and unexpectedly, that the use of a cosmetic composition comprising more than 1% by weight, relative to the total weight of the composition, of at least one volatile linear alkane, at least one solid fatty alcohol and at least one polymeric thickener containing sugar units makes it possible to overcome the drawbacks mentioned above and to improve significantly the cosmetic properties of the hair such as the smoothness, tonicity, disentangling, lightness and suppleness.

In particular, during the application of the composition and after rinsing, smoother, more supple hair is obtained.

Furthermore, this use leads especially to wet hair that is easier to disentangle, lighter and more tonic, and to dried hair that is more supple and that has a smoother feel.

Thus, one subject of the invention is the use, for the cosmetic treatment of keratin fibres such as the hair, of a cosmetic composition comprising, in a cosmetically acceptable medium, more than 1% by weight, relative to the total weight of the composition, of at least one volatile linear alkane, at least one solid fatty alcohol and at least one polymeric thickener containing sugar units.

According to the invention, a cosmetic composition comprising, in a cosmetically acceptable medium:
more than 1% by weight, relative to the total weight of the composition, of one or more volatile linear alkanes,
one or more solid fatty alcohols and
one or more polymeric thickeners containing sugar units

is used for the cosmetic treatment of keratin fibres, such as the hair. This use leads especially to the production of better smoothing, tonicity, disentangling, lightness and/or suppleness properties.

The described composition also makes up a part of the invention.

Another subject of the invention is a cosmetic process for treating keratin materials, preferably keratin fibres such as the hair, using the composition.

The term “cosmetically acceptable medium” and “cosmetic composition” means a medium or composition that is compatible with keratin materials and in particular the hair.

The cosmetically acceptable medium preferably comprises water or a mixture of water and of a cosmetically acceptable solvent chosen from C1-C4 alcohols, such as ethanol, isopropanol, tert-butanol or n-butanol; polyols such as glycerol, propylene glycol and polyethylene glycols; and mixtures thereof.

The term “one or more volatile linear alkane(s)” means, without preference, “one or more volatile linear alkane oil(s)”.

A volatile linear alkane that is suitable for use in the invention is liquid at room temperature (about 25° C.) and at atmospheric pressure (101 325 Pa or 760 mmHg).

The term “volatile linear alkane that is suitable for use in the invention” means a linear alkane that can evaporate on contact with the skin in less than one hour, at room temperature (25° C.) and atmospheric pressure (101 325 Pa), which is liquid at room temperature, especially having an evaporation rate ranging from 0.01 to 15 mg/cm2/minute, at room temperature (25° C.) and atmospheric pressure (101 325 Pa).

Preferably, the volatile linear alkane(s) that are suitable for use in the invention have an evaporation rate ranging from 0.01 to 3.5 mg/cm2/minute and better still from 0.01 to 1.5 mg/cm2/minute, at room temperature (25° C.) and atmospheric pressure (101 325 Pa).

More preferably, the volatile linear alkane(s) that are suitable for use in the invention have an evaporation rate ranging from 0.01 to 0.8 mg/cm2/minute, preferentially from 0.01 to 0.3 mg/cm2/minute and even more preferentially from 0.01 to 0.12 mg/cm2/minute, at room temperature (25° C.) and atmospheric pressure (101 325 Pa).

The evaporation rate of a volatile alkane in accordance with the invention (and more generally of a volatile solvent) may especially be evaluated by means of the protocol described in WO 06/013 413, and more particularly by means of the protocol described below.

15 g of volatile hydrocarbon-based solvent are placed in a crystallizing dish (diameter: 7 cm) placed on a balance that is in a chamber of about 0.3 m3 with regulated temperature (25° C.) and hygrometry (50% relative humidity).

The volatile hydrocarbon-based solvent is allowed to evaporate freely, without stirring it, while providing ventilation by means of a fan (Papst-Motoren, reference 8550 N, rotating at 2700 rpm) placed in a vertical position above the crystallizing dish containing the volatile hydrocarbon-based solvent, the blades being directed towards the crystallizing dish, 20 cm away from the bottom of the dish.

The mass of volatile hydrocarbon-based solvent remaining in the crystallizing dish is measured at regular time intervals.

The evaporation profile of the solvent is then obtained by plotting the curve of the amount of product evaporated (in mg/cm2) as a function of the time (in minutes).

The evaporation rate is then calculated, which corresponds to the tangent to the origin of the curve obtained. The evaporation rates are expressed as mg of volatile solvent evaporated per unit surface area (cm2) and per unit of time (minutes).

According to one preferred embodiment, the volatile linear alkanes that are suitable for use in the invention have a non-zero vapour pressure (also known as the saturating vapour pressure), at room temperature, in particular a vapour pressure ranging from 0.3 Pa to 6000 Pa.

Preferably, the volatile linear alkane(s) that are suitable for use in the invention have a vapour pressure ranging from 0.3 to 2000 Pa and better still from 0.3 to 1000 Pa, at room temperature (25° C.).

More preferably, the volatile linear alkane(s) that are suitable for use in the invention have a vapour pressure ranging from 0.4 to 600 Pa, preferentially from 1 to 200 Pa and even more preferentially from 3 to 60 Pa, at room temperature (25° C.).

According to one embodiment, a volatile linear alkane that is suitable for use in the invention may have a flash point that is within the range from 30 to 120° C. and more particularly from 40 to 100° C. The flash point is in particular measured according to standard ISO 3679.

According to one embodiment, the volatile linear alkanes that are suitable for use in the invention may be linear alkanes comprising from 7 to 15 carbon atoms, preferably from 8 to 14 carbon atoms and better still from 9 to 14 carbon atoms.

More preferably, the volatile linear alkanes that are suitable for use in the invention may be linear alkanes comprising from 10 to 14 carbon atoms and even more preferentially from 11 to 14 carbon atoms.

A volatile linear alkane that is suitable for use in the invention may advantageously be of plant origin.

Preferably, the volatile linear alkane or the mixture of volatile linear alkanes present in the composition according to the invention comprises at least one 14C (carbon-14) carbon isotope. In particular, the 14C isotope may be present in a numerical isotope ratio 14C/12C (or 14C/12C ratio) of greater than or equal to 1×10−16, preferably greater than or equal to 1×10−15, more preferably greater than or equal to 7.5×10−14 and better still greater than or equal to 1.5×10−13. Preferably, the ratio 14C/12C ranges from 6×10−13 to 1.2×10−12.

The amount of 14C isotopes in the volatile linear alkane or the mixture of volatile linear alkanes may be determined via methods known to those skilled in the art such as the Libby counting method, liquid scintillation spectrometry or accelerator mass spectrometry.

Such an alkane may be obtained, directly or in several steps, from a plant raw material, such as an oil, a butter, a wax, etc.

As examples of alkanes that are suitable for use in the invention, mention may be made of the alkanes described in patent applications WO 2007/068 371 and WO 2008/155 059. These alkanes are obtained from fatty alcohols, which are themselves obtained from coconut oil or palm oil.

As examples of volatile linear alkanes that are suitable for use in the invention, mention may be made of n-heptane (C7), n-octane (C8), n-nonane (C9), n-decane (C10), n-undecane (C11), n-dodecane (C12), n-tridecane (C13), n-tetradecane (C14) and n-pentadecane (C15), and mixtures thereof. According to one particular embodiment, the volatile linear alkane is chosen from n-nonane, n-undecane, n-dodecane, n-tridecane and n-tetradecane, and mixtures thereof.

According to one preferred embodiment, mention may be made of mixtures of n-undecane (C11) and of n-tridecane (C 13) obtained in Examples 1 and 2 of patent application WO 2008/155 059.

Mention may also be made of n-dodecane (C12) and n-tetradecane (C14) sold, respectively, under the references Parafol 12-97 and Parafol 14-97 by the company Sasol, and also mixtures thereof.

One embodiment consists in using only one volatile linear alkane.

Alternatively, a mixture of at least two different volatile linear alkanes, differing from each other by a carbon number n of at least 1, in particular differing from each other by a carbon number of 1 or 2, may be used.

According to one embodiment, a mixture of at least two different volatile linear alkanes comprising from 10 to 14 carbon atoms and differing from each other by a carbon number of at least 1 is used. Examples that may especially be mentioned include mixtures of C10/C11, C11/C12 or C12/C13 volatile linear alkanes.

According to another embodiment, a mixture of at least two different volatile linear alkanes comprising from 10 to 14 carbon atoms and differing from each other by a carbon number of at least 2 is used. Examples that may especially be mentioned include mixtures of C10/C12 or C12/C14 volatile linear alkanes, for an even carbon number n, and the C11/C13 mixture for an odd carbon number n.

According to one preferred embodiment, a mixture of at least two different volatile linear alkanes comprising from 10 to 14 carbon atoms and differing from each other by a carbon number of at least 2, and in particular a mixture of C11/C13 volatile linear alkanes or a mixture of C12/C14 volatile linear alkanes, is used.

Other mixtures combining more than two volatile linear alkanes according to the invention, for instance a mixture of at least three different volatile linear alkanes comprising from 7 to 15 carbon atoms and differing from each other by a carbon number of at least 1, may be used in the invention.

In the case of mixtures of two volatile linear alkanes, the two volatile linear alkanes preferably represent more than 95% and better still more than 99% by weight of the mixture.

According to one particular mode of the invention, in a mixture of volatile linear alkanes, the volatile linear alkane having the smaller carbon number is predominant in the mixture.

According to another mode of the invention, a mixture of volatile linear alkanes in which the volatile linear alkane having the larger carbon number is predominant in the mixture used.

As examples of mixtures that are suitable for use in the invention, mention may be made especially of the following mixtures:

from 50% to 90% by weight, preferably from 55% to 80% by weight and more preferentially from 60% to 75% by weight of Cn volatile linear alkane with n ranging from 7 to 15,

from 10% to 50% by weight, preferably from 20% to 45% by weight and preferably from 24% to 40% by weight of Cn+x volatile linear alkane with x greater than or equal to 1, preferably x=1 or x=2, with n+x between 8 and 14,

relative to the total weight of alkanes in the mixture.

In particular, the mixture of volatile linear alkanes may also contain:

less than 2% by weight and preferably less than 1% by weight of branched hydrocarbons,

and/or less than 2% by weight and preferably less than 1% by weight of aromatic hydrocarbons,

and/or less than 2% by weight, preferably less than 1% by weight and preferentially less than 0.1% by weight of unsaturated hydrocarbons,

the percentages being expressed relative to the total weight of the mixture.

More particularly, the volatile linear alkanes that are suitable for use in the invention may be used in the form of an n-undecane/n-tridecane mixture.

In particular, a mixture of volatile linear alkanes will be used comprising:

from 55% to 80% by weight and preferably from 60% to 75% by weight of C11 volatile linear alkane (n-undecane) and

from 20% to 45% by weight and preferably from 24% to 40% by weight of C13 volatile linear alkane (n-tridecane),

relative to the total weight of alkanes in the mixture.

According to one particular embodiment, the mixture of alkanes is an n-undecane/n-tridecane mixture. In particular, such a mixture may be obtained according to Example 1 or Example 2 of patent application WO 2008/155 059.

According to another particular embodiment, the n-dodecane sold under the reference Parafol 12-97 by Sasol is used.

According to another particular embodiment, the n-tetradecane sold under the reference Parafol 14-97 by Sasol is used.

According to yet another embodiment, a mixture of n-dodecane and n-tetradecane is used.

The volatile linear alkane(s) are used in an amount strictly greater than 1% by weight relative to the total weight of the composition, and preferably in an amount ranging from 1.1% to 90% by weight, in particular from 1.1% to 50% by weight, more particularly from 1.5% to 20% by weight and better still from 2% to 10% by weight relative to the total weight of the composition.

For the purposes of the present invention, the term “solid fatty alcohol” means a compound of formula R—OH, R denoting a linear or branched C10-C30 alkyl or alkenyl chain, which is solid at room temperature (25° C.) and at atmospheric pressure (101 325 Pa).

Preferably, R denotes a linear C10-C30 alkyl chain.

The solid fatty alcohol(s) used in the invention are preferably linear C 12-30 and better still C12-26 fatty alcohols.

As examples of solid fatty alcohols that may be used in the present invention, mention may be made especially of myristyl alcohol, cetyl alcohol, stearyl alcohol, cetearyl alcohol, arachidyl alcohol, behenyl alcohol, lignoceryl alcohol, ceryl alcohol and montanyl alcohol, and mixtures thereof.

Preferably, the solid fatty alcohol used in the invention is chosen from myristyl alcohol, cetyl alcohol, stearyl alcohol, cetearyl alcohol and behenyl alcohol, and mixtures thereof.

The solid fatty alcohol(s) may be used in an amount ranging from 0.5% to 20% by weight, preferably from 1% to 15% by weight and more particularly from 2% to 10% by weight relative to the total weight of the composition.

For the purposes of the present invention, the term “polymeric thickener” means a polymer which, when introduced to a proportion of 1% by weight in an aqueous solution or an aqueous-alcoholic solution containing 30% ethanol, and at pH 7, makes it possible to achieve a viscosity of at least 0.1 Pa·s (100 cps) and preferably at least 0.5 Pa·s (500 cps), at 25° C. and at a shear rate of 1 s−1. This viscosity may be measured using a cone/plate viscometer (Haake R600 rheometer or the like).

For the purposes of the present invention, the term “sugar unit” means a unit derived from a carbohydrate of formula Cn(H2O)n−1 or (CH2O)n, which may be optionally modified by substitution, and/or by oxidation and/or by dehydration.

The sugar units that may be included in the chemical formula of the polymeric thickeners of the invention are preferably derived from the following sugars:

glucose

galactose

arabinose

rhamnose

mannose

xylose

fucose

anhydrogalactose

galacturonic acid

glucuronic acid

mannuronic acid

galactose sulfate

anhydrogalactose sulfate.

The polymeric thickeners containing sugar units of the invention may be natural or synthetic polysaccharides.

They may be nonionic, anionic, amphoteric or cationic.

As polymeric thickeners containing sugar units used in the invention, mention may be made especially of:
native gums such as:

a) tree and shrub exudates, including:

gum arabic (branched polymer of galactose, arabinose, rhamnose and glucuronic acid)

ghatti gum (polymer derived from arabinose, galactose, mannose, xylose and glucuronic acid)

karaya gum (polymer derived from galacturonic acid, galactose, rhamnose and glucuronic acid)

gum tragacanth (or tragacanth) (polymer of galacturonic acid, galactose, fucose, xylose and arabinose)

b) polymers derived from algae, including:

agar (polymer derived from galactose and anhydrogalactose)

alginates (polymers of mannuronic acid and glucuronic acid)

carrageenans and furcellerans (polymers of galactose sulfate and anhydrogalactose sulfate)

c) gums derived from seeds or tubers, including:

guar gum (polymer of mannose and galactose)

locust bean gum (polymer of mannose and galactose)

fenugreek gum (polymer of mannose and galactose)

tamarind gum (polymer of galactose, xylose and glucose)

konjac gum (polymer of glucose and mannose)

starch (glucose polymer)

d) microbial gums, including:

xanthan gum (polymer of glucose, mannose acetate, mannose/pyruvic acid and glucuronic acid)

gellan gum (polymer of partially acylated glucose, rhamnose and glucuronic acid)

scleroglucan gum (glucose polymer)

e) plant extracts, including:

cellulose (glucose polymer, cellobiose repeating unit)

starch (glucose polymer, amylose and amylopectin repeating units).

These native gums may be physically or chemically modified. A physical treatment that may especially be mentioned is heat.

Chemical treatments that may be mentioned include esterification, etherification, amidation and oxidation reactions. These treatments lead to polymers that may be nonionic, anionic, cationic or amphoteric, and preferably nonionic or cationic.

Preferably, these chemical or physical treatments are applied to guar gums, locust bean gums, starches and celluloses.

In one embodiment of the invention, the polymeric thickener containing sugar units is chosen from nonionic guar gums modified with C1-C6 hydroxyalkyl groups, guar gums modified with cationic groups, locust bean gum modified with hydroxypropyltrimonium groups, pregelatinized and/or oxidized and/or crosslinked and/or esterified starches, amphoteric starches, cellulose ethers, cellulose esters and cellulose ester ethers, and mixtures thereof.

The nonionic guar gums that may be used according to the invention may be modified with C1-C6 hydroxyalkyl groups.

Among the hydroxyalkyl groups that may be mentioned, for example, are hydroxymethyl, hydroxyethyl, hydroxypropyl and hydroxybutyl groups.

These guar gums are well known in the prior art and can be prepared, for example, by reacting the corresponding alkene oxides, for instance propylene oxides, with the guar gum so as to obtain a guar gum modified with hydroxypropyl groups.

The degree of hydroxyalkylation, which corresponds to the number of alkylene oxide molecules consumed by the number of free hydroxyl functions present on the guar gum, preferably ranges from 0.4 to 1.2.

Such nonionic guar gums optionally modified with hydroxyalkyl groups are sold, for example, under the trade names Jaguar HP8, Jaguar HP60, Jaguar HP105 and Jaguar HP120 by the company Rhodia Chimie.

The guar gums modified with cationic groups that may more particularly be used according to the invention are guar gums comprising trialkylammonium cationic groups. Preferably, 2% to 30% by number of the hydroxyl functions of these guar gums bear trialkylammonium cationic groups. Even more preferentially, 5% to 20% of the number of hydroxyl functions of these guar gums bear trialkylammonium cationic groups.

Among these trialkylammonium groups, mention may be made most particularly of trimethylammonium and triethylammonium groups.

Even more preferentially, these groups represent from 5% to 20% by weight relative to the total weight of the modified guar gum.

According to the invention, guar gums modified with 2,3-epoxypropyltrimethylammonium chloride may be used.

These guar gums modified with cationic groups are products that are already known per se and are described, for example, in U.S. Pat. No. 3,589,578 and U.S. Pat. No. 4,0131,307. Such products are moreover sold especially under the trade names Jaguar C13S, Jaguar C 15 and Jaguar C17 by the company Rhodia Chimie.

A modified locust bean gum that may be used is the cationic locust bean gum containing hydroxypropyltrimonium groups, such as Catinal CLB 200 sold by the company Toho.

The starch molecules used in the present invention may be botanically derived from cereals or tubers. Thus, the starches are chosen, for example, from corn starch, rice starch, cassava starch, barley starch, potato starch, wheat starch, sorghum starch and pea starch.

The starches may be chemically or physically modified, especially via one or more of the following reactions: pregelatinization, oxidation, crosslinking, esterification, etherification, amidation, heat treatments.

More particularly, these reactions may be performed in the following manner:
pregelatinization by splitting the starch granules (for example drying and cooking in a drying drum);
oxidation with strong oxidizing agents leading to the introduction of carboxyl groups into the starch molecule and to depolymerization of the starch molecule (for example by treating an aqueous starch solution with sodium hypochlorite);
crosslinking with functional agents capable of reacting with the hydroxyl groups of the starch molecules, which will thus bind together (for example with glyceryl and/or phosphate groups);
esterification in alkaline medium for the grafting of functional groups, especially C1-C6 acyl (acetyl) and C1-C6 hydroxyalkyl groups such as hydroxyethyl and hydroxypropyl, carboxymethyl or octenylsuccinic.

Monostarch phosphates (of the type Am-O—PO—(OX)2), distarch phosphates (of the type Am-O—PO—(OX)—O-Am) or even tristarch phosphates (of the type Am-O—PO—(O-Am)2) or mixtures thereof, Am meaning starch, may especially be obtained by crosslinking with phosphorus compounds.

X especially denotes alkali metals (for example sodium or potassium), alkaline-earth metals (for example calcium or magnesium), ammonium salts, amine salts such as those of monoethanolamine, diethanolamine, triethanolamine or 3-amino-1,2-propanediol, and ammonium salts derived from basic amino acids such as lysine, arginine, sarcosine, ornithine or citrulline.

The phosphorus compounds may be, for example, sodium tripolyphosphate, sodium orthophosphate, phosphorus oxychloride or sodium trimetaphosphate.

Use will preferentially be made of distarch phosphates, in particular hydroxypropyl distarch phosphates, or of compounds rich in distarch phosphate, especially hydroxypropyl distarch phosphate, for instance the product sold under the references Prejel VA-70-T AGGL (gelatinized hydroxypropyl cassava distarch phosphate) or Prejel TK1 (gelatinized cassava distarch phosphate) or Prejel 200 (gelatinized acetyl cassava distarch phosphate) by the company Avebe, or Structure Zea from National Starch (gelatinized corn distarch phosphate).

A preferred starch is a starch that has undergone at least one chemical modification, such as at least one esterification.

According to the invention, it is also possible to use amphoteric starches, these amphoteric starches comprising one or more anionic groups and one or more cationic groups. The anionic and cationic groups may be attached to the same reactive site of the starch molecule or to different reactive sites; they are preferably attached to the same reactive site. The anionic groups may be of carboxylic, phosphate or sulfate type, preferably of carboxylic type. The cationic groups may be of primary, secondary, tertiary or quaternary amine type.

The amphoteric starches are especially chosen from the compounds having the following formulae:

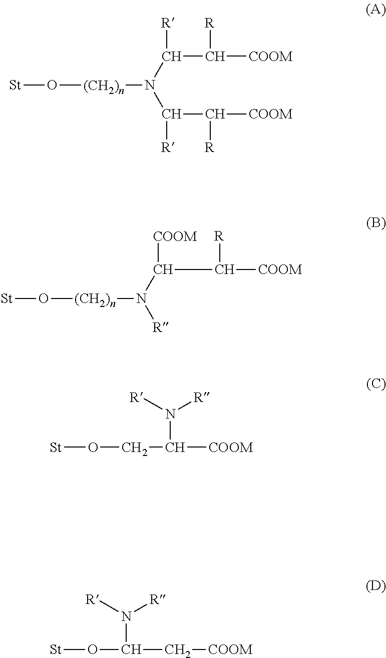

in which formulae:

St-O represents a starch molecule,

R, which may be identical or different, represents a hydrogen atom or a methyl radical,

R′, which may be identical or different, represents a hydrogen atom, a methyl radical or a group —COOH,

n is an integer equal to 2 or 3,

M, which may be identical or different, denotes a hydrogen atom, an alkali metal or alkaline-earth metal such as Na, K or Li, NH4, a quaternary ammonium or an organic amine,

R″ represents a hydrogen atom or an alkyl radical containing from 1 to 18 carbon atoms.

These compounds are especially described in U.S. Pat. No. 5,455,340 and U.S. Pat. No. 4,017,460.

The starch molecules may be derived from any botanical source of starch, especially such as corn, potato, oat, rice, tapioca, sorghum, barley or wheat. Hydrolysates of the starches mentioned above may also be used. The starch is preferably derived from potato.

The starches of formula (B) or (C) are particularly used. Starches modified with 2-chloroethylaminodipropionic acid, i.e. the starches of formula (B) or (C) in which R, R′, R″ and M represent a hydrogen atom and n is equal to 2, are more particularly used. A preferred amphoteric starch is a starch chloroethylamidodipropionate.

Cellulose derivatives such as modified celluloses may also be used as polymeric thickener containing sugar units. They may be anionic, cationic, amphoteric or nonionic.

The cellulose derivatives may be cellulose ethers, cellulose esters or cellulose ester ethers.

Among the cellulose esters are inorganic esters of cellulose (cellulose nitrates, sulfates, phosphates, etc.), organic cellulose esters (cellulose monoacetates, triacetates, amidopropionates, acetatebutyrates, acetatepropionates and acetatetrimellitates, etc.) and mixed organic/inorganic esters of cellulose, such as cellulose acetatebutyrate sulfates and cellulose acetatepropionate sulfates. Among the cellulose ester ethers, mention may be made of hydroxypropylmethylcellulose and ethylcellulose sulfates.

Among the nonionic cellulose ethers that may be mentioned are alkylcelluloses such as methylcelluloses and ethylcelluloses (for example Ethocel Standard 100 Premium from Dow Chemical); hydroxyalkylcelluloses such as hydroxymethylcelluloses and hydroxyethylcelluloses (for example Natrosol 250 HHR sold by Aqualon) and hydroxypropylcelluloses (for example Klucel EF from Aqualon); mixed hydroxyalkyl-alkylcelluloses such as hydroxypropyl-methylcelluloses (for example Methocel E4M from Dow Chemical), hydroxyethyl-methylcelluloses, hydroxyethyl-ethylcelluloses (for example Bermocoll E 481 FQ from Akzo Nobel) and hydroxybutyl-methylcelluloses.

Among the anionic cellulose ethers, mention may be made of carboxyalkylcelluloses and salts thereof. Examples that may be mentioned include carboxymethylcelluloses, carboxymethylmethylcelluloses (for example Blanose 7M from the company Aqualon) and carboxymethylhydroxyethylcelluloses and the sodium salts thereof.

Among the cationic cellulose ethers that may be mentioned are crosslinked or non-crosslinked quaternized hydroxyethylcelluloses. The quaternizing agent may especially be diallyldimethylammonium chloride (for example Celquat L200 from National Starch). Another cationic cellulose ether that may be mentioned is hydroxypropyltrimethylammonium hydroxyethyl cellulose (for example Ucare Polymer JR 400 from Amerchol).

As other polymeric thickeners that may be used in the invention, mention may be made of associative polymers chosen especially from chemically modified celluloses or guar gums. Examples of chemically modified celluloses are:
celluloses or derivatives thereof, modified with groups comprising at least one fatty chain, such as alkyl, arylalkyl or alkylaryl groups or mixtures thereof in which the alkyl groups are of C8-C22;
nonionic alkylhydroxyethylcelluloses such as the products Natrosol Plus Grade 330 CS and Polysurf 67 (C16 alkyl) sold by the company Aqualon;
quaternized alkylhydroxyethylcelluloses (cationic), such as the products Quatrisoft LM 200, Quatrisoft LM-X 529-18-A, Quatrisoft LM-X 529-18-B (C12 alkyl) and Quatrisoft LM-X 529-8 (C18 alkyl) sold by the company Amerchol, the products Crodacel QM and Crodacel QL (C12 alkyl) and Crodacel QS (C18 alkyl) sold by the company Croda, and the product Softcat SL 100 sold by the company Amerchol;
nonionic nonoxynylhydroxyethylcelluloses such as the product Amercell HM-1500 sold by the company Amerchol;
nonionic alkylcelluloses such as the product Bermocoll EHM 100 sold by the company Berol Nobel.

Associative guar derivatives that may be used include hydroxypropyl guars modified with a fatty chain, such as the product Esaflor HM 22 (modified with a C22 alkyl chain) sold by the company Lamberti; the product Miracare XC 95-3 (modified with a C14 alkyl chain) and the product RE 205-146 (modified with a C20 alkyl chain) sold by Rhodia Chimie.

The polymeric thickener(s) containing sugar units used in the invention are preferably chosen from guar gums, starches and celluloses, which may or may not be modified.

Preferably, the polymeric thickeners containing sugar units of the invention are nonionic or cationic.

The polymeric thickener(s) containing sugar units are preferably used in an amount ranging from 0.01% to 10% by weight, even more preferentially from 0.1% to 5% by weight and better still from 0.2% to 3% by weight relative to the weight of the composition.

The composition used in the present invention may also contain at least one cationic surfactant.

Examples of cationic surfactants that may especially be mentioned include salts of optionally polyoxyalkylenated primary, secondary or tertiary fatty amines; quaternary ammonium salts; imidazoline derivatives; or amine oxides of cationic nature.

As an example of a salt of an optionally polyoxyalkylenated primary, secondary or tertiary fatty amine, mention may be made especially of distearylamine.

Examples of quaternary ammonium salts that may especially be mentioned include:
those having the general formula (I) below:

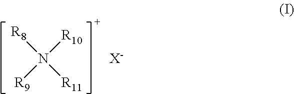

in which the radicals R8 to R11, which may be identical or different, represent a linear or branched aliphatic radical comprising from 1 to 30 carbon atoms or an aromatic radical such as aryl or alkylaryl, at least one of the radicals R8 to R11 comprising from 8 to 30 carbon atoms and preferably from 12 to 24 carbon atoms. The aliphatic radicals may comprise heteroatoms especially such as oxygen, nitrogen, sulfur and halogens. The aliphatic radicals are chosen, for example, from alkyl, alkoxy, polyoxy(C2-C6)alkylene, alkylamide, (C12-C22)alkylamido(C2-C6)alkyl, (C12-C22)alkylacetate and hydroxyalkyl radicals, comprising from about 1 to 30 carbon atoms; X— is an anion chosen from the group of halides, phosphates, acetates, lactates, (C2-C6)alkyl sulfates, alkyl sulfonates and alkylaryl sulfonates.

Among the quaternary ammonium salts of formula (1), the ones preferably used are, on the one hand, tetraalkylammonium chlorides, for instance dialkyldimethylammonium chlorides or alkyltrimethylammonium chlorides, in which the alkyl radical comprises from about 12 to 22 carbon atoms, in particular behenyl-trimethylammonium chloride, distearyldimethylammonium chloride, cetyltrimethyl-ammonium chloride, or benzyldimethylstearylammonium chloride, or, on the other hand, palmitylamidopropyltrimethylammonium chloride or stearamidopropyldimethyl(myristyl acetate)ammonium chloride sold under the name Ceraphyl® 70 by the company Van Dyk.
quaternary ammonium salts of imidazoline, for instance those of formula (II) below:

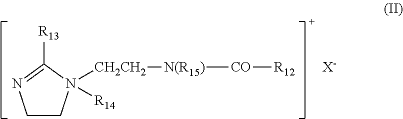

in which R12 represents an alkenyl or alkyl radical comprising from 8 to 30 carbon atoms, for example fatty acid derivatives of tallow, R13 represents a hydrogen atom, a C1-C4 alkyl radical or an alkenyl or alkyl radical comprising from 8 to 30 carbon atoms, R14 represents a C1-C4 alkyl radical, R15 represents a hydrogen atom or a C1-C4 alkyl radical, and X— is an anion chosen from the group of halides, phosphates, acetates, lactates, alkyl sulfates, alkyl sulfonates and alkylaryl sulfonates. R12 and R13 preferably denote a mixture of alkenyl or alkyl radicals comprising from 12 to 21 carbon atoms, for example fatty acid derivatives of tallow, R14 denotes a methyl radical and R15 denotes a hydrogen atom. Such a product is sold, for example, under the name Rewoquat® W75 by the company Rewo;
the diquaternary ammonium salts of formula (III):

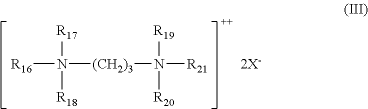

in which R16 denotes an aliphatic radical comprising from about 16 to 30 carbon atoms, R17, R18, R19, R20 and R21, which may be identical or different, are chosen from hydrogen and an alkyl radical comprising from 1 to 4 carbon atoms, and X— is an anion chosen from the group of halides, acetates, phosphates, nitrates and methyl sulfates. Such diquaternary ammonium salts especially comprise propanetallowdiammonium dichloride;
quaternary ammonium salts containing at least one ester function, such as those of formula (IV) below:

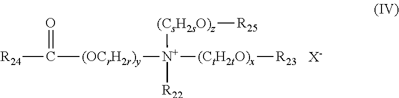

in which:

R22 is chosen from C1-C6 alkyl radicals and C1-C6 hydroxyalkyl or dihydroxyalkyl radicals;

R23 is chosen from:
a radical

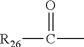

linear or branched, saturated or unsaturated C1-C22 hydrocarbon-based radicals R27,
a hydrogen atom,

R25 is chosen from:
a radical

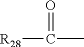

linear or branched, saturated or unsaturated C1-C6 hydrocarbon-based radicals R29,
a hydrogen atom,

R24, R26 and R28, which may be identical or different, are chosen from linear or branched, saturated or unsaturated C7-C21 hydrocarbon-based radicals;

r, s and t, which may be identical or different, are integers ranging from 2 to 6;

y is an integer ranging from 1 to 10;

x and z, which may be identical or different, are integers ranging from 0 to 10;

X— is a simple or complex, organic or mineral anion;

with the proviso that the sum x+y+z is from 1 to 15, that when x is 0, then R23 denotes R27 and that when z is 0, then R25 denotes R29.

The alkyl radicals R22 may be linear or branched, but more particularly linear.

R22 preferably denotes a methyl, ethyl, hydroxyethyl or dihydroxypropyl radical, and more particularly a methyl or ethyl radical.

Advantageously, the sum x+y+z is from 1 to 10.

When R23 is a hydrocarbon-based radical R27, it may be long and may contain from 12 to 22 carbon atoms, or may be short and may contain from 1 to 3 carbon atoms.

When R25 is a hydrocarbon-based radical R29, it preferably contains 1 to 3 carbon atoms.

Advantageously, R24, R26 and R28, which may be identical or different, are chosen from linear or branched, saturated or unsaturated C11-C21 hydrocarbon-based radicals, and more particularly from linear or branched, saturated or unsaturated C11-C21 alkyl and alkenyl radicals.

Preferably, x and z, which may be identical or different, are equal to 0 or 1.

Advantageously, y is equal to 1.

Preferably, r, s and t, which may be identical or different, are equal to 2 or 3, and even more particularly are equal to 2.

The anion is preferably a halide (chloride, bromide or iodide) or an alkyl sulfate, more particularly methyl sulfate. However, methanesulfonate, phosphate, nitrate, tosylate, an anion derived from an organic acid, such as acetate or lactate, or any other anion that is compatible with the ammonium containing an ester function, may be used.

The anion X— is even more particularly chloride or methyl sulfate.

Use is made more particularly in the composition according to the invention of the ammonium salts of formula (IV) in which:
R22 denotes a methyl or ethyl radical,
x and y are equal to 1;
z is equal to 0 or 1;
r, s and t are equal to 2;
R23 is chosen from:
   a radical

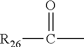

methyl, ethyl or C14-C22 hydrocarbon-based radicals,
   a hydrogen atom;
R25 is chosen from:
   a radical

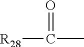

a hydrogen atom;
R24, R26 and R28, which may be identical or different, are chosen from linear or branched, saturated or unsaturated C13-C17 hydrocarbon-based radicals and preferably from linear or branched, saturated or unsaturated C13-C17 alkyl and alkenyl radicals.

The hydrocarbon-based radicals are advantageously linear. Examples that may be mentioned include the compounds of formula (IV) such as the diacyloxyethyl-dimethylammonium, diacyloxyethylhydroxyethylmethylammonium, monoacyloxyethyldihydroxyethylmethylammonium, triacyloxyethylmethylammonium and monoacyloxyethylhydroxyethyldimethylammonium salts (chloride or methyl sulfate in particular), and mixtures thereof. The acyl radicals preferably contain 14 to 18 carbon atoms and are obtained more particularly from a plant oil such as palm oil or sunflower oil. When the compound contains several acyl radicals, these radicals may be identical or different.

These products are obtained, for example, by direct esterification of triethanolamine, triisopropanolamine, an alkyldiethanolamine or an alkyldiisopropanolamine, which are optionally oxyalkylenated, with fatty acids or with fatty acid mixtures of plant or animal origin, or by transesterification of the methyl esters thereof. This esterification is followed by a quaternization using an alkylating agent such as an alkyl halide (preferably a methyl or ethyl halide), a dialkyl sulfate (preferably dimethyl or diethyl sulfate), methyl methanesulfonate, methyl para-toluenesulfonate, glycol chlorohydrin or glycerol chlorohydrin.

Such compounds are sold, for example, under the names Dehyquart® by the company Henkel, Stepanquat® by the company Stepan, Noxamium® by the company CECA or Rewoquat® WE 18 by the company Rewo-Witco.

The cationic surfactants that are particularly preferably used in the invention are chosen from quaternary ammonium salts such as behenyltrimethylammonium chloride, quaternary ammonium salts containing at least one ester function, such as distearoylethylhydroxyethylmethylammonium methosulfate, and mixtures thereof.

When at least one of the cationic surfactants is used, it (they) may be used in an amount ranging from 0.1% to 10% by weight, preferably from 0.2% to 8% by weight and more particularly from 0.2% to 5% by weight relative to the total weight of the composition.

In one embodiment of the invention, the composition may also comprise one or more plant oils. As examples of plant oils that may be used in the invention, mention may be made especially of camellina oil, sweet almond oil, avocado oil, castor oil, olive oil, jojoba oil, sunflower oil, wheatgerm oil, sesame oil, groundnut oil, grapeseed oil, soybean oil, rapeseed oil, safflower oil, coconut oil, corn oil, hazelnut oil, shea butter, palm oil, apricot kernel oil and beauty-leaf oil.

When at least one of the plant oils is present, it (they) may be used in an amount ranging from 0.01% to 30% by weight, preferably from 0.1% to 20% by weight and more particularly from 0.2% to 10% by weight relative to the total weight of the composition.

According to one embodiment of the invention, the composition may also comprise at least one silicone.

The silicones that may be used in accordance with the invention may be soluble or insoluble in the composition. They may in particular be polyorganosiloxanes that are insoluble in the composition of the invention and may be in the form of oils, waxes, resins or gums.

The insoluble silicones are especially dispersed in the compositions in the form of particles generally having a number-average size of between 2 nanometres and 100 micrometres and preferably between 20 nanometres and 20 micrometres (measured with a granulometer).

The organopolysiloxanes are defined in greater detail in Walter Noll's Chemistry and Technology of Silicones (1968) Academic Press. They can be volatile or non-volatile.

When they are volatile, the silicones are more particularly chosen from those having a boiling point of between 60° C. and 260° C., and even more particularly from:

(i) cyclic silicones containing from 3 to 7 and preferably 4 to 5 silicon atoms. These are, for example, octamethylcyclotetrasiloxane sold in particular under the name Volatile Silicone 7207 by Union Carbide or Silbione 70045 V 2 by Rhodia, decamethylcyclopentasiloxane sold under the name Volatile Silicone 7158 by Union Carbide, and Silbione 70045 V 5 by Rhodia, and mixtures thereof.

Mention may also be made of cyclocopolymers of the dimethylsiloxane/methylalkylsiloxane type, such as Volatile Silicone FZ 3109 sold by the company Union Carbide, having the chemical structure:

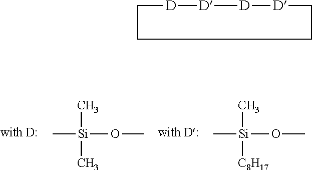

Mention may also be made of mixtures of cyclic silicones with organosilicon compounds, such as the mixture of octamethylcyclotetrasiloxane and tetratrimethylsilyl-pentaerythritol (50/50) and the mixture of octamethylcyclotetrasiloxane and oxy-1,1′-bis-(2,2,2′,2′,3,3′-hexatrimethylsilyloxy)neopentane;

(ii) linear volatile silicones containing 2 to 9 silicon atoms and having a viscosity of less than or equal to 5×10−6 m2/s at 25° C. An example is decamethyltetrasiloxane sold in particular under the name SH 200 by the company Toray Silicone. Silicones belonging to this category are also described in the article published in Cosmetics and Toiletries, Vol. 91, Jan. 76, pp. 27-32, Todd & Byers Volatile Silicone Fluids for Cosmetics.

Among the non-volatile silicones that may especially be mentioned are polyalkylsiloxanes, polyarylsiloxanes, polyalkylarylsiloxanes, silicone gums and resins, polyorganosiloxanes modified with organofunctional groups, polysiloxane(A)-polyoxyalkylene(B) linear block copolymers of (A-B)ₙ type with n>3; grafted silicone polymers, with a non-silicone organic backbone, consisting of an organic main chain formed from organic monomers not comprising silicone, onto which are grafted, within the chain and also optionally on at least one of its ends, at least one polysiloxane macromonomer; grafted silicone polymers, with a polysiloxane backbone grafted with non-silicone organic monomers, comprising a polysiloxane main chain onto which are grafted, within the chain and also optionally on at least one of its ends, at least one organic macromonomer not comprising silicone; and also mixtures thereof.

Examples of polyalkylsiloxanes that may especially be mentioned include polydimethylsiloxanes containing trimethylsilyl end groups with a viscosity of from 5×10−6 to 2.5 m2/s at 25° C. and preferably 1×10−5 to 1 m2/s. The viscosity of the silicones is measured, for example, at 25° C. according to ASTM standard 445 Appendix C.

Among these polyalkylsiloxanes, mention may be made, in a non-limiting manner, of the following commercial products:
the Silbione oils of the 47 and 70 047 series or the Mirasil oils sold by Rhône-Poulenc, for instance the oil 70 047 V 500 000;
the oils of the Mirasil series sold by the company Rhône-Poulenc;
the oils of the 200 series from the company Dow Corning, such as, more particularly, DC200 with a viscosity of 60 000 cSt;
the Viscasil oils from General Electric and certain oils of the SF series (SF 96, SF 18) from General Electric.

Mention may also be made of polydimethylsiloxanes containing dimethylsilanol end groups (Dimethiconol according to the CTFA name) such as the oils of the 48 series from the company Rhone-Poulenc.

In this category of polyalkylsiloxanes, mention may also be made of the products sold under the names Abil Wax 9800 and 9801 by the company Goldschmidt, which are poly(C1-C20)alkylsiloxanes.

The polyalkylarylsiloxanes are chosen particularly from linear and/or branched polydimethylmethylphenylsiloxanes or polydimethyldiphenylsiloxanes, with a viscosity of from 1×10−5 to 5×10−2 m2/s at 25° C.

Among these polyalkylarylsiloxanes, mention may be made, by way of example, of the products sold under the following names:
the Silbione oils of the 70 641 series from Rhône-Poulenc;
the oils of the Rhodorsil 70 633 and 763 series from Rhône-Poulenc;
the oil Dow Corning 556 Cosmetic Grade Fluid from Dow Corning;
the silicones of the PK series from Bayer, such as the product PK20;
the silicones of the PN and PH series from Bayer, such as the products PN1000 and PH1000;
certain oils of the SF series from General Electric, such as SF 1023, SF 1154, SF 1250, SF 1265.

The silicone gums that can be used in accordance with the invention are, in particular, polydiorganosiloxanes having high number-average molecular masses of between 200 000 and 1 000 000, used alone or as a mixture in a solvent. This solvent can be chosen from volatile silicones, polydimethylsiloxane (PDMS) oils, polyphenylmethylsiloxane (PPMS) oils, isoparaffins, polyisobutylenes, methylene chloride, pentane, dodecane and tridecane, or mixtures thereof.

Mention may be made more particularly of the following products:
polydimethylsiloxane gums,
polydimethylsiloxane/methylvinylsiloxane gums,
polydimethylsiloxane/diphenylsiloxane gums,
polydimethylsiloxane/phenylmethylsiloxane gums,
polydimethylsiloxane/diphenylsiloxane/methylvinylsiloxane gums.

Silicones that can be used in the composition in accordance with the invention are mixtures such as:
mixtures formed from a polydimethylsiloxane hydroxylated at the chain end (referred to as dimethiconol according to the nomenclature in the CTFA dictionary) and from a cyclic polydimethylsiloxane (referred to as cyclomethicone according to the nomenclature in the CTFA dictionary), such as the product Q2 1401 sold by the company Dow Corning;
mixtures formed from a polydimethylsiloxane gum with a cyclic silicone, such as the product SF 1214 Silicone Fluid from the company General Electric; this product is an SF 30 gum corresponding to a dimethicone, having a number-average molecular weight of 500 000, dissolved in the oil SF 1202 Silicone Fluid corresponding to decamethylcyclopentasiloxane;
mixtures of two PDMSs of different viscosities, and more particularly of a PDMS gum and a PDMS oil, such as the product SF 1236 from the company General Electric. The product SF 1236 is a mixture of an SE 30 gum defined above, having a viscosity of 20 m2/s, and an SF 96 oil, with a viscosity of 5×10−6 m2/s. This product preferably comprises 15% SE 20 gum and 85% SF 96 oil.

The organopolysiloxane resins that can be used in accordance with the invention are crosslinked siloxane systems containing the following units: R2SiO2/2, R3SiO1/2, RSiO3/2 and SiO4/2 in which R represents a hydrocarbon-based group containing 1 to 16 carbon atoms or a phenyl group.

Among these products, those particularly preferred are the ones in which R denotes a C1-C4 lower alkyl radical, more particularly methyl, or a phenyl radical.

Among these resins, mention may be made of the product sold under the name Dow Corning 593 or those sold under the names Silicone Fluid SS 4230 and SS 4267 by the company General Electric, which are silicones of dimethyl/trimethyl siloxane structure.

Mention may also be made of the trimethyl siloxysilicate type resins sold in particular under the names X22-4914, X21-5034 and X21-5037 by the company Shin-Etsu.

The organomodified silicones that can be used in accordance with the invention are silicones as defined above and containing in their structure one or more organofunctional groups attached via a hydrocarbon-based group.

Among the organomodified silicones, mention may be made of polyorganosiloxanes comprising:
polyethyleneoxy and/or polypropyleneoxy groups optionally comprising C6-C24 alkyl groups, such as the products known as dimethicone copolyol sold by the company Dow Corning under the name DC 1248 or the oils Silwet® L 722, L 7500, L 77 and L 711 by the company Union Carbide, and the (C12)alkylmethicone copolyol sold by the company Dow Corning under the name Q2 5200;
substituted or unsubstituted amine groups, such as the products sold under the name GP 4 Silicone Fluid and GP 7100 by the company Genesee, or the products sold under the names Q2 8220 and Dow Corning 929 or 939 by the company Dow Corning. The substituted amine groups are, in particular, C1-C4 aminoalkyl groups;
quaternary ammonium groups, for instance the products sold under the names Abilquat 3272 and Abilquat 3474 by the company Goldschmidt;
thiol groups such as the products sold under the names GP 72 A and GP 71 from Genesee;
alkoxylated groups such as the product sold under the name Silicone Copolymer F-755 by SWS Silicones and Abil Wax® 2428, 2434 and 2440 by the company Goldschmidt;
hydroxylated groups such as the polyorganosiloxanes containing a hydroxyalkyl function, described in French patent application FR-A-85 16334;
acyloxyalkyl groups such as, for example, the polyorganosiloxanes described in U.S. Pat. No. 4,957,732;
anionic groups of carboxylic acid type, such as, for example, in the products described in patent EP 186 507 from the company Chisso Corporation, or of alkylcarboxylic type, such as those present in the product X-22-3701 E from the company Shin-Etsu; 2-hydroxyalkyl sulfonate; 2-hydroxyalkyl thiosulfate such as the products sold by the company Goldschmidt under the names Abil® S201 and Abil® S255;
hydroxyacylamino groups, such as the polyorganosiloxanes described in patent application EP 342 834. Mention may be made, for example, of the product Q2-8413 from the company Dow Corning.

The silicones that are particularly preferred in the invention are unsubstituted polydimethylsiloxanes and amino silicones.

When at least one of the silicones is present, it is (they are) preferably contained in an amount ranging from 0.05% to 20% by weight, more particularly from 0.1% to 10% by weight and better still from 0.2% to 5% by weight relative to the total weight of the composition.

The composition used in the invention may also comprise one or more natural compounds or compounds of natural origin, such as natural active agents and extracts, for instance fruit juices, essential oils and plant extracts.

The term “natural compound” means a product that is obtained from the earth or the soil, or from plants or animals, optionally via one or more physical processes, for instance milling, refining, distillation, purification or filtration.

The term “compound of natural origin” means a natural compound that has undergone one or more chemical treatments that do not affect the essential qualities of this compound and/or a compound predominantly comprising natural compounds.

The composition used in the invention may also comprise one or more standard additives that are well known in the art, such as anionic, nonionic or amphoteric surfactants, anionic, cationic, nonionic or amphoteric polymers, polyols, proteins, vitamins, reducing agents, plasticizers, softeners, antifoams, moisturizers, pigments, clays, mineral fillers, UV-screening agents, mineral colloids, peptizers, solubilizers, fragrances, preserving agents, nacreous agents, propellants and mineral thickeners, or mixtures thereof.

A person skilled in the art will take care to select the optional additives and the amount thereof such that they do not harm the properties of the compositions used in the present invention.

These additives are generally present in the composition according to the invention in an amount ranging from 0 to 20% by weight relative to the total weight of the composition.

The compositions used in the invention may be in the form of a more or less thickened lotion, a cream, a gel or an emulsion.

The invention also relates to a cosmetic process for treating keratin materials, preferably keratin fibres such as the hair, which consists in applying an effective amount of a cosmetic composition as described above to the materials, and optionally in rinsing it out after an optional leave-in time.

When the composition according to the invention is applied in the form of a lotion or a cream, it is optionally left to stand on the hair for about 30 seconds to 5 minutes, and is then optionally rinsed out with water.

The examples that follow are given as illustrations of the present invention.

In the examples that follow, all the amounts are indicated as weight percentages of active material relative to the total weight of the composition, unless otherwise indicated.

### EXAMPLES

### Example 1

Compositions A and B below were prepared from the ingredients indicated in the table below.

| | Compositions | |
|---|---|---|
| | A | B |
| | (comparative) | (invention) |
| Cetearyl alcohol(1) | 3 | 3 |
| Cetyl esters (myristyl/cetyl/stearyl | 0.5 | 0.5 |
| myristate/palmitate/stearate mixture)(2) | | |
| Distearoylethylhydroxyethylmethyl- | 3 | 3 |
| ammonium methosulfate/cetearyl | | |
| alcohol mixture (75/25 by weight)(3) | | |
| Apricot kernel oil | 1 | 1 |
| Corn starch (Zea mays starch)(4) | 0.5 | 0.5 |
| Mixture predominantly formed from n- | — | 3 |
| undecane and n-tridecane(5) | | |
| Preserving agents | 0.35 | 0.35 |
| Fragrance | 0.6 | 0.6 |
| Water qs | 100 | 100 |

(1)sold under the trade name Lanette O OR by the company Cognis
(2)sold under the trade name Miraceti by the company Laserson
(3)sold under the trade name Dehyquart F 75 by the company Cognis
(4)sold under the trade name C*Gel 04201 by the company Cargill
(5)according to Example 2 of WO 2008/155 059

Compositions A and B were applied to the hair, and the properties obtained were compared. Remarkably better smoothing on rinsing and on wet hair was observed with composition B, compared with the properties observed for the comparative composition A.

In addition, the hair is more tonic and lighter on rinsing and on wet hair with composition B according to the invention.

### Example 2

Compositions C and D below were prepared from the ingredients indicated in the table below.

| | Compositions | |
|---|---|---|
| | C | D |
| Cetearyl alcohol(1) | 3 | 3 |
| Distearoylethylhydroxyethyldimethylammonium | 4 | — |
| methosulfate/cetearyl alcohol (75/25 by weight)(2) | | |
| Behenyltrimethylammonium chloride at 80% | — | 2 |
| active material | | |
| Amodimethicone as a 60% cationic aqueous | — | 1.5 |
| emulsion(3) | | |
| Hydroxyethylcellulose(4) | 0.2 | — |
| Hydroxypropyl guar(5) | — | 0.3 |
| Mixture mainly formed from n-undecane and n- | — | 2 |
| tridecane(6) | | |
| Mixture of n-dodecane and n-tetradecane(7) | 6 | — |
| Preserving agents | 0.35 | 0.35 |
| Fragrance | 0.6 | 0.6 |
| Water qs | 100 | 100 |

(1)sold under the trade name Lanette O OR by the company Cognis
(2)sold under the trade name Dehyquart F 75 by the company Cognis
(3)sold under the trade name Dow Corning 2-8299 Cationic Emulsion by the company Dow Corning
(4)sold under the trade name Natrosol 250 HHR by the company Aqualon
(5)sold under the trade name Jaguar HP 105 by the company Rhodia
(6)according to Example 2 of WO 2008/155 059
(7)sold under the trade name Vegelight 1214 by the company Biosynthis

Compositions C and D were applied to the hair. Excellent smoothing on rinsing and on wet hair was observed. In addition, the hair showed excellent tonicity and lightness on rinsing and on wet hair.

The above written description of the invention provides a manner and process of making and using it such that any person skilled in this art is enabled to make and use the same, this enablement being provided in particular for the subject matter of the appended claims, which make up a part of the original description.

As used herein, the words “a” and “an” and the like carry the meaning of “one or more.”

The phrases “selected from the group consisting of,” “chosen from,” and the like include mixtures of the specified materials. Terms such as “contain(s)” and the like are open terms meaning ‘including at least’ unless otherwise specifically noted.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein. In this regard, certain embodiments within the invention may not show every benefit of the invention, considered broadly.

1. A method for the treatment of keratin fibres, comprising applying to keratin fibers a composition comprising, in a cosmetically acceptable medium, more than 1% by weight, relative to the total weight of composition, of one or more volatile linear alkanes, one or more solid fatty alcohols and one or more polymeric thickeners containing sugar units.

2. The method according to claim 1, wherein the volatile linear alkane is a linear alkane comprising from 7 to 15 carbon atoms.

3. The method according to claim 1, wherein the volatile linear alkane is chosen from n-heptane (C7), n-octane (C8), n-nonane (C9), n-decane (C 10), n-undecane (C11), n-dodecane (C12), n-tridecane (C13), n-tetradecane (C14) and n-pentadecane (C15), and mixtures thereof.

4. The method according to claim 1, wherein the volatile linear alkane is chosen from n-nonane, n-undecane, n-dodecane, n-tridecane and n-tetradecane, and mixtures thereof.

5. The method according to claim 1, wherein the volatile linear alkane is of plant origin.

6. The method according to claim 1, wherein the volatile linear alkane(s) are present in an amount ranging from 1.1% to 90% by weight relative to the total weight of the composition.

7. The method according to claim 1, wherein the solid fatty alcohol is a linear C 12-30 fatty alcohol.

8. The method according to claim 1, wherein the solid fatty alcohol is chosen from myristyl alcohol, cetyl alcohol, stearyl alcohol, cetearyl alcohol, arachidyl alcohol, behenyl alcohol, lignoceryl alcohol, ceryl alcohol and montanyl alcohol, and mixtures thereof, and preferably from myristyl alcohol, cetyl alcohol, stearyl alcohol, cetearyl alcohol and behenyl alcohol, and mixtures thereof.

9. The method according to claim 1, wherein the solid fatty alcohol(s) are present in a content of 0.5% to 20% by weight relative to the total weight of the composition.

10. The method according to claim 1, wherein the polymeric thickener containing sugar units is a natural or synthetic polysaccharide.

11. The method according to claim 1, wherein the polymeric thickener containing sugar units is chosen from:
a) tree or shrub exudates,
b) gums derived from algae,
c) gums derived from seeds or tubers,
d) microbial gums,
e) plant extracts,
which are unmodified or physically or chemically modified.

12. The method according to claim 1, wherein the polymeric thickener containing sugar units is chosen from guar gums, locust bean gums, starches and celluloses, which have been physically or chemically modified.

13. The method according to claim 1, wherein the polymeric thickener containing sugar units is nonionic, cationic, anionic or amphoteric.

14. The method according to claim 1, wherein the polymeric thickener containing sugar units is chosen from nonionic guar gums modified with C1-C6 hydroxyalkyl groups, guar gums modified with cationic groups, locust bean gum modified with hydroxypropyltrimonium groups, pregelatinized and/or oxidized and/or crosslinked and/or esterified starches, amphoteric starches, cellulose ethers, cellulose esters and cellulose ester ethers, and mixtures thereof.

15. The method according to claim 1, wherein the polymeric thickener containing sugar units is an associative polymer.

16. The method according to claim 1, wherein the polymeric thickener(s) containing sugar units are present in a content of 0.01% to 10% by weight relative to the total weight of the composition.

17. The method according to claim 1, wherein the cosmetic composition further comprises at least one cationic surfactant.

18. The method according to claim 17, wherein the cationic surfactant(s) are present in a content of 0.1% to 10% by weight relative to the total weight of the composition.

19. The method according to claim 1, wherein the composition further comprises at least one plant oil chosen from camellina oil, sweet almond oil, avocado oil, castor oil, olive oil, jojoba oil, sunflower oil, wheatgerm oil, sesame oil, groundnut oil, grapeseed oil, soybean oil, rapeseed oil, safflower oil, coconut oil, corn oil, hazelnut oil, shea butter, palm oil, apricot kernel oil and beauty-leaf oil, and mixtures thereof.

20. The method according to claim 1, wherein the composition further comprises at least one silicone.

21. The method according to claim 1, wherein the keratin fibres are hair.
